(19) United States
(12) Patent Application Publication
(10) Pub. No.: US 2012/0129712 A1
(43) Pub. Date: May 24, 2012

(54) ANTIBODIES DIRECTED TO TRA ANTIGENS, AND METHODS OF PRODUCTION, SCREENING AND ANALYSIS OF SAID ANTIBODIES, AS WELL AS METHODS OF ANALYSIS OF STEM CELLS AND CANCER CELL

(75) Inventors: Tero Satomaa, Helsinki (FI); Suvi Natunen, Vantaa (FI); Leena Valmu, Helsinki (FI); Jari Natunen, Vantaa (FI)

(73) Assignees: GLYKOS FINLAND OY, Helsinki (FI); SUOMEN PUNAINEN RISTI, VERIPALVELU, Helsinki (FI)

(21) Appl. No.: 13/266,090

(22) PCT Filed: Apr. 26, 2010
(86) PCT No.: PCT/FI2010/050340

§ 371 Date: Nov. 15, 2011

(30) Foreign Application Priority Data
Apr. 24, 2009 (FI) 20095459

(51) Int. Cl. C40B 30/04; C40B 40/10; G01N 33/53; C07K 17/10

(52) U.S. Cl. 506/9; 530/391.1; 506/18; 436/501; 435/7.1; 435/7.21; 435/7.23

(57) ABSTRACT

A complex of Tra-antibody bound to an isolated glycan comprising type I—N-acetyllactosamine comprising target structure, and methods and uses utilizing said complex.

### BACKGROUND OF THE INVENTION

### Tra-1-81 and Tra-1-60 Antibodies

Tra-1-81 and -60 antibodies, referred here as Tra-antibodies have been used as a standard marker for embryonic stem cells. The antibodies have been known useful for the characterization of mesenchymal stem cells or differentiated emsenchymal stem cells. Tra-1-81 and -60 have been reported to recognize a carbohydrate epitopes on podocalyxin, which has been considered to be a keratan sulphate epitope and a sialylated keratan sulphate, respectively (Schopperle and DeWolf (2007) Stem Cells 25(3) 723-730), Keratan sulphate is usually sulphated two lactosamine [(SE-6)ₙGalβ4(SE-6)ₘGlcNAcβ3]ₚ, with possible terminal sialic acid or midcahin fucose derivatization. The glycans bound by Tra-1-81 or -60 have not been structurally characterized. The present invention revealed unusual non-modified type I N-acetyllactosamine specificity for Tra-1-81 and -60 antibodies. The invention is directed to use of the novel specifity for production or analysis of novel Tra-antibodies.

### DESCRIPTION OF THE INVENTION

Present inventors analyzed data from glycan array profile analysis of novel N-acetyllactosamine Type I-II tetraose epitope, Galβ1-3GlcNAcβ1-3Galβ1-4GlcNAc, binding antibodies, such as certain Tra-antibodies, and specific clones thereof and revealed novel specificity for the antibody. The best 50 target glycans for of TRA-1-81 and Tra-1-60 antibodies are shown in Table 1 and Table 2 respectively

The invention is directed to methods to support the analysis of various cells or tissues or other glycan comprising materials by Type I-II tetraose epitope, Galβ1-3GlcNAcβ1-3Galβ1-4GlcNAc, binding antibodies, such as Tra-antibodies, and further optionally controlling the binding, and controlled methods for producing novel Tra-antibody clones. It is realized, that to produce a new Type I-II tetraose epitope antibody, such as a Tra-antibody, it would be useful to assay the new antibodies against the target glycan structures according to the invention and optionally further against the control glycans.

### Novel Type I-II N-acetyllactosamine Tetraose Epitope Specifities of Tra-1-60 and Tra-1-81

Previously known type I N-acetyllactosamine specific antibodies are specific for corresponding non-reducing end disaccharides or trisaccharides. The antibodies typically bind to glycolipid structure Galβ1-3GlcNAcβ1-3Galβ1-4GlcβCer, including certain K21 antibody clones. In contrast, present invention revealed unusual Poly-N-acetyllactosamine type specificity, including reducing end GlcNAc residue in tetrasaccharide epitope, comprising type I-II tetraose epitope, Galβ1-3GlcNAcβ1-3Galβ1-4GlcNAc, which can be further bound to polylactosamine or other glycan structure, in a preferred embodiment by β1-6 linkage. Preferred oligosaccharides and their conjugates include the saccharides 130 and 379 (Consortium for Functional Glycomics, array glycan numbers) and their conjugates. Preferred additional control saccharides for antibody specificity control are with the same or similar structures include ones listed in the above for Tra-1-81 and isomeric to the target structures and reducing end elongated and/or sialylated or fucosylated oligosaccharide sequence variants thereof, from the Tables 1 and 2, especially when the binding specificity includes the type I-II tetraose, Galβ1-3GlcNAcβ1-3Galβ1-4GlcNAc, core epitope.

Type I-II tetraose binding antibody of invention is also referred such as Tra-antibody/Tra-specificty, which includes also preferred examples of the antibodies namely Tra-1-81 and Tra-1-60 and most preferably the clones used in examples, most preferably of Tra-1-60.

The present invention is directed to use of the novel type I-II N-acetyllactosamine tetraose specificty such as specificity of the Tra-antibodies for the analysis of presence the non-reducing end tetrasaccharide epitope Galβ1-3GlcNAcβ1-3Galβ1-GlcNAc(R) or more preferably Galβ1-3GlcNAcβ1-3Galβ1-GlcNAcβ or structure according to Formula 1 Galβ1-3GlcNAcβ1-3Galβ1-GlcNAc(β)_{b}(CH₂)ₓR, wherein R is a organic conjugate or residue including spacer and carbohydrate, b is 0 or 1, and x is an integer from 0 or 20, preferably 1 to 16, more preferably 1 to 10. More preferably Galβ1-3GlcNAcβ1-3Galβ1-GlcNAcβ6Gal(NAc) or Galβ1-3GlcNAcβ1-3Galβ1-GlcNAcβSp, wherein Sp is an organic spacer. In preferred embodiment the antibodies are used for the analysis of the structures in a material expected to contain the structures.

Accordingly, the invention is especially directed to a complex of an antibody and an isolated glycan comprising a target structure Galβ1-3GlcNAcβ1-3Galβ1-GlcNAc or target structure according to Formula 1. The invention is especially directed to the complexes when the antibody is not capable of binding non-binding control structure Galβ1-3GlcNAcβ1-3Galβ1-Glc and/or one or several of the other control structures of the invention.

The invention is further directed to arrays comprising the complex of the invention and optionally non-binding saccharides of the invention essentially not in complex with the antibody.

The conjugate (R) is preferably linked to i) a polymer such as carbohydrate, polysaccharide (agarose, cellulose, chitosan, dextran, glycosaminoglycan etc), protein such as albumins, KLH (Keyhole limpet hemocyanin), transferrin, or organic polymer such as polyacrylamide or polyether (e.g. Polyethyleneglycol-derivative) or ii) detectable label such as a fluorescent molecule (fluorescein, Alexa fluor etc.), or selectively non-covalently binding molecule such biotin, or analog or multifluoroalkyl or a nucleotide or an antigen iii) further immobilizable organic molecule such as a lipid including hydrophobic alkyl, and aromatic organic molecules comprising preferably more than 10, even more preferably more than 15 carbon atoms, such as C10-30 alkyl or arylalkyl alcohols or fatty acids or amines iv) conjugate is a spacer linking the glycan epitope, preferably through a spacer, to a solid phase such as a plastic, glass or metal surface including microarray plate/matrix, microtiter plate well, gold surface including surface Plasmon resonance. Preferred spacers include e.g. spacer comprising C1-10 alkyls and arylalkyls, and bifunctional forms of molecules in iii) or spacer of the arrays of examples and published analogous array spacers, bifunctional means comprising at least two conjugateble atom or atom group such as amine, alkohol, carboxylic acid, aldehyde, ketone, hydrazide, amino-oxy, alkylamino-oxy, thiol, maleimide, alkyneand azide. The conjugateble atoms or groups are selected so that counterpart of one conjugateble atom/group is conjugateble to reducing end or reducing end derivative such as Ser/Thr/peptide derivative of the saccharide epitope and one conjugateble atom/group is conjugateble to the solid phase, e.g. by amide, oxime, thiol-malemide, aldehyde/ketone-hydrazide, alkyne-azide product, or ester linkage.

There are numerous published commercial protein and saccharide polymer conjugates and synthesis technologies available. In a preferred embodiment the conjugate is formed by a glycosidic linkage, preferably O—, N—, C— or S— glycosidic bond, more preferably an alfa-glycosidic bond. The preferred conjugate or spacer structure may include a Glycan-amino acid or Glyco-peptide epitope such as OSβ6GalNAc serine or threonine residue being glycosidically alfa-linked to the reducing end of the glycan epitope (Type I-II tetraose-OS), as in natural O-glycans.

Binding in present invention means specific binding recognizing the bound saccharides effectively and essentially not recognizing non-bound saccharides. The binding in a preferred embodiment is measured as a solid phase assay, e.g. as in examples. The essentially non-binding preferably means less than average 50% signal of the signals of best (preferably average of one or the two best in Tables 1 or 2 for the respective antibody specificity) binding saccharides, more preferably less than on average 35%, even more preferably less than 20%, even more preferably less than 10%, and most preferably less than 5%. In a preferred embodiment the invention is directed to optimized practically exclusive or exclusive binding specificity with non binding signals less than 4% even more preferably less than 3 and most preferably less than 2% of the signals of the best binding saccharides.

The saccharides/glycans/oligosaccharide(s) epitope or structure mean oligosaccharide epitopes described, these are preferably non-reducing end oligosaccharide sequences which are not modified by any monosaccharide structures except optionally from the reducing end. Preferably the oligosaccharide epitopes are reducing end conjugates and/or free oligosaccharides. The saccharides are in a specific embodiment optionally modified by a chemical derivative smaller than monosaccharides to hydroxyls.

Non-binding variants

The non-binding or essentially non-binding related structures include tetrasaccharides with reducing end Glc: Galβ1-3GlcNAcβ1-3Galβ1-4Glc (no 131, consortium for glycobiology glycan array glycan numbering), with two type 1 N-acetyllactosamines Galβ1-3GlcNAcβ1-3Galβ1-3GlcNAc (no 280), isomer with (36-linkage with lactosamines Galβ1-3GlcNAcβ1-6Galβ1-4GlcNAc (no 403), or two type II LacNAcs Galβ1-4GlcNAcβ1-3Galβ1-4GlcNAc (no 146), variant with reducing end Glc and sialic acid on position 6 of subterminal GlcNAc Galβ1-3(Neu5Acα6)GlcNAcβ1-3Galβ1-4Glc (no 126), α3-sialylated variant Neu5Acα3Galβ1-3GlcNAcβ1-3Galβ1-4GlcNAc (no 223), sialylated and difucosylated derivative Neu5Acα3Galβ1-3(Fucα4)GlcNAcβ1-3Galβ1-4(Fucα3)GlcNAc (no 217), trisaccharide lacking non-reducing end Gal, GlcNAcβ1-3Galβ1-4GlcNAc (no 163 and 164), or non-reducing end disaccharide Galβ3GlcNAc (nos 132 and 133) had much weaker binding or practically no binding.

The most characteristic non-binding structure is Galβ1-3GlcNAcβ1-3Galβ1-4Glc, the invention is especially directed to methods and complexes of the invention, when the antibody binds to Galβ1-3GlcNAcβ1-3Galβ1-4GlcNAc and not to Galβ1-3GlcNAcβ1-3Galβ1-4Glc, and optionally or/further not to other non-binding structures.

The invention revealed the preferred antibody in the novel complex structures and novel methods when the antibody does not bind to modified tetrasaccharide derivatives/structures according to

(T)ₚGalβ1-3(Fucα4)ₙGlcNAcβ1-3Galβ1-Glc(NAc)ₘR, Formula 2

wherein p, n and m are integers 0 or 1 independently and/or the larger reducing end elongated or conjugated target oligosaccharide sequences, the fucose residue in ( ) indicated by n is a branch in the structure, and T is terminal monosaccharide residue, preferably a sialic acid such as Neu5Acα3-structure linked to non-reducing end Gal, and R is reducing end derivative or conjugate as defined above with the provisio that that when m is 1 either p or n is also 1 (the structure is not the binding epitope Galβ1-3GlcNAcβ1-3Galβ1-4GlcNAc).

The invention further revealed the preferred antibody in the novel complex structures and novel methods when the antibody does not bind to modified tetrasaccharide derivatives/structures according to

(T)ₚGalβ1-z(Fucα4)ₙGlcNAcβ1-yGalβ1-qGlc(NAc)ₘR, Formula 3

wherein p, n and m are integers 0 or 1 independently and/or the larger reducing end elongated or conjugated target oligosaccharide sequences, the fucose residue in ( ) indicated by n is a branch in the structure, and T is terminal monosaccharide residue, preferably a sialic acid such as Neu5Acα3-structure linked to non-reducing end Gal; and z, y and q are linkage positions selected from the group 3, 4, or 6, preferably q and z being 3 or 4, independently and y is 3 or 6, with the provisio that when m is 1, and z and y are 3, and q is 4, either p or n is also 1 (the structure is not the binding epitope Galβ1-3GlcNAcβ1-3Galβ1-4GlcNAc).

Arrays of saccharide epitopes binding and non-binding with antibodies

Preferably, said complex is in an array of glycan structures, and optionally the array comprises said saccharides the antibody is capable of binding, and optionally further said saccharides the antibody is not capable of binding. The preferred array comprises preferably Galβ1-3GlcNAcβ1-3Galβ1-4Glc as non-binding saccharide or/and further at least one, more preferably at least two, even more preferably at least three and most preferably all saccahrides according to the Formula 2 or in separate embodiment according to the Formula 3. Said glycan array can be a solid phase conjugated saccharide array or an array of detectable/marked glycans in solution. The invention is directed to array composition comprising the preferred binding saccharide epitope and at least one preferred non-binding saccharide epitope.

The invention further revealed several non-binding control glycans, preferably as listed in Tables 1 and 2, which are further preferred as non-binding saccahrides in the array The invention is directed to binding assays such as solid phase assays involving binding of the antibody the glycan or chemical synthetic conjugate of the glycan. The invention is especially directed to the method for detection of the novel Type 1-3 tetraose epitope by preferred antibodies according of the invention and optionally validating the binding by using an array according to the invention, or a method using a binding and at least one non-binding saccharide(s) as a control of the.

Preferably the solid phase assay or liquid phase assay involves binding of the antibody to the oligosaccharide glycan or chemical synthetic conjugate or solid phase conjugate of the glycan.

In preferred embodiment the antibodies with the novel specificity are in complex with a synthetic glycan group or a glycan array comprising preferred binding target and binding or non-binding control structures. The combination/complex substance may in a form of a glycan array device or a kit including the antibody and the array, e.g an array and antibody(ies) as indicated in examples. In the combination substance the preferred antibody is combined with or bound to the binding structures.

### Inhibition of the Complexes of the Invention

The invention is further directed to combinations of the antibodies and the saccharide epitopes of the invention wherein the saccharide (s) are used as soluble oligosaccharides or conjugates optionally to measure the interaction in solution or to inhibit binding of the antibody to a target saccharide and further optionally to control saccharide when saccharides are solid phase conjugated as described by the invention or cell/tissue surface conjugated. Typically 0.001 nM to 1 mM, more preferably 1 nM to 100 microM inhibitor calculated as monovalent saccharide of the invention is used as soluble saccharides preferably as inhibitors.

### Novel Complex of Type I-II Tetraose Antibody Such as, Tra-Antibody and Tetrasacchride Epitope Comprising Glycans for Binding and Inhibition Assay

The invention is directed to a complex of a (preferably new) antibody with Tra-binding specificity, with a type I N-acetyllactosamine (Galβ3GlcNAc) epitope comprising glycan. In a preferred embodiment the antibody has optimized binding activity to the target tetrasaccharide. It is realized that the complex may have been occurred in assay with natural cells or podocalyxin protein, but not with isolated purified components and the idea of the ligand glycans is clearly teaching away from the present invention. The present invention is directed to novel complexes with novel synthetic or isolated Galβ3GlcNAc comprising structures, more preferably Galβ3GlcNAc, such as
a) a free oligosaccharide or part thereof, or
b) an oligosaccharide or synthetic chemical conjugate thereof, or
c) a natural or biosynthetic structure enriched with regard to Galβ3GlcNAc structures, and preferably depleted with other glycan stuctures from the natural source.

Preferred natural sources includes human cells according to the invention and in a preferred embodiment a podocalyxin protein with increased Tra-antibody binding Galβ3GlcNAc structures.

In another embodiment the amount of the glycan structure is standardized in the natural material with regard to the Galβ3GlcNAc-stucture.

The Tra-antibodies are preferably optimized for recognition of Tra-type structures on human cells known to contain Tra-ligands, bound by known Tra-antibodies, most preferably from human embryonic stem cells, and human mesenchymal cells including mesenchymal stem cells and differentiated variants thereof. Human cells known to contain Tra-ligands are bound by known Tra-antibodies, preferably Tra-1-60 or Tra-1-81. In a preferred embodiment the antibodies are optimized by methods according to the invention for the recognition of most of or all of the cells

The target epitope of the novel antibody binding, and saccharide part of the novel complex is an oligosaccharide sequence according to the Formula 1b Galβ1-3(Fucα4)ₘGlcNAcβ1-3Galβ1-Glc(NAc)ₙ, wherein n and m are integers 0 or 1 respectively and/or the larger reducing end elongated target oligosaccharide sequences, wherein preferably n is 1.

The invention is under preferred embodiments directed to complex of Lewis a variants of Tra-antibodies wherein n is 1 and m is 0 or 1; for complex of LNT-type variants wherein m is 0 or 1, and n is 0; or more preferably N-acetyllactosamine structure wherein both n and m are 1. The oligosaccharide sequence is preferably a non-reducing end terminal oligosaccharide sequence.

The invention revealed that the Tra-antibodies can be complexed with or bound to an isolated glycan comprising the tetrasaccharide target structure according to the invention. The invention is directed to binding assays such as solid phase assays involving binding of the antibody the glycan or chemical synthetic conjugate of the glycan.

The invention is preferably directed to the solid phase assay or liquid phase assay involving binding of the antibody the oligosaccharide glycan or chemical synthetic conjugate of the glycan.

### Validation Method

The invention is especially directed to the use of the tetrasaccharide comprising control material for validation of the analysis of the antibody binding to cells or other biological materials. In a preferred embodiment the control material is purified a oligosaccharide preferably conjugated to a solid surface or to control cells in a solid phase assay or used as a soluble inhibitor or soluble analyst (e.g. labelled conjugate for a fluorescence polarization assay) to validate the binding specificity of the antibody.

In a preferred embodiment the invention is directed to Tra-antibody analysis kit comprising the tetrasaccharide sequence comprising glycan or glycoconjugate or a cell sample optimized with the glycan structure expression, preferably for the validation of the analysis of Tra-structure in cells or tissues. In a preferred embodiment, the invention is directed to a test kit including instructions for the analysis of the novel target structure, preferably as defined in Formula I, and optionally instructions for reporting the amount of the novel target structure.

In further preferred embodiment specific α3-(or α6-)sialidase enzyme is used to optimized the presense of the Tra antigens on cell surface and a specific β3-galactosidase is used to reduce the amount of the structure on cells. Co-pending patent applications of the applicants describe the glycosidase reactions of the structures.

### Antibody Selection and Production Method

The invention is directed to a method of selection of a new antibody with Tra-specificity, preferably with optimized binding activity to the target tetrasaccharide epitopes of Formula I.

The invention is directed to the production of variants:

for production of Lewis a variants of Tra-antibodies wherein m is 1 and n is 0 or 1;

for production of LNT-type variants wherein m is 0 or 1, and n is 1, in preferred embodiment m is 0; or

most preferably N-acetyllactosamine structure wherein both n and m are 0.

It is realized that rational production of the Tra-antibodies has been impossible because the exact antigen structure is not known. The antibody producing cells/animal may die or get compromised and there is need to get similar or specificity optimised antibodies with a Tra-antibody specificity. The present invention is in a preferred embodiment directed to the rational production of new antibodies with Tra-specificity. The novel method includes steps of
1) providing a sample comprising at least one antibody (or functional antibody fragment binding to an antigen). In preferred embodiment in form of serum or a phage display library.
2) contacting the sample with a glycan structure comprising terminal non-reducing end terminal oligosaccharide sequence Galβ1-3GlcNAcβ1-3Galβ1-GlcNAc
3) measuring the binding of the antibody to the oligosaccharide sequence.
4) optionally contacting the antibody sample with at least one preferred control glycan structure, more preferably with two, three, four of most preferably at lest five control glycan structures.
5) optionally selecting antibody with specific binding to the target structures but low or non-existent binding to specificity control saccharides, or in a specific embodiment selecting antibodies with additionally or specifically corresponding Lewis a specificity.
6) optionally using an oligosaccharide sequence comprising the terminal non-reducing end tetrasaccharide sequence or being the tetrasaccharide for the inhibition of the binding of the antibody the oligosaccharide sequence,
7) optionally using β3-galactosidase of α3- and or α6-sialidase enzymes to optimize or reduce the amount of the antibody target structures on cells.

The invention is especially directed to the use of optimization of the binding activity of a Tra-type antibody using the novel tetrasaccharide sequence.

The invention is further directed to the use of the produced antibodies or the validation methods for the analysis

for the analysis of stem cells or cancer cells or other cells or tissues known to bind to Tra-antibodies, preferably human embryonic type stem cells, including embryonic stem cells e.g as described in PCT/FI 2008/050018 and pluripotent equivalents thereof such as IPS cells, induced pluripotent cells (Yamanaka et al) or mesenchymal stem cells or osteogenically or adipocyte differentiated mesenchymal stem cells as described in PCT/FI 2008/050019. The inventors has studied ESC by the methods of the application and revealed potential glycans structures comprising type I-II tetraose β6-linked on GalNAcα on core II O-glycan. The structure is cleavable by β3-galactosidase and endo-beta-galactosidase (e.g. B. fragilis or E. freundii), which are preferred control enzymes when assaying biological samples with the enzymes and verifying or validating complexes with natural glcyans, preferably from stem cells.

The Tra-antibodies or Tra-type antibodies includes antibodies with binding specificity essentially similar to Tra-1-60 and/or Tra-1-80. The Tra-antibodies include the known Tra-antibodies and potential ones obtainable or analyzable by the methods according to the invention. The new Tra-antibodies does not include the known antibodies with Tra-1-60 and/or Tra-1-80 specificity. The Tra-antibodies bind specifically and preferably practically exclusively to glycan structures according to Formula I. In a preferred embodiment the binding of the antibodies are similar as on Tables 1 and 2, when measured by a solid phase assay similar to the micro array analysis, preferably showing several fold (preferably 3 or 4-fold) higher signals, more preferably at least 10-fold, and even more preferably more than 100-fold binding signals for a target structure or target structures according to Formula I compared to control structures. In a preferred embodiment the antibody specificity is optimized for higher specificity than the original antibodies for the preferred target structures of Formula I.

### A Novel Assay Method

In a preferred embodiment the present invention is directed to assay of biological materials including the reporting of the amount of novel target glycan, preferably including steps
a) providing a biological material
b) contacting the material with a Tra-antibody
c) providing a report indicating an amount of the novel Tra-target glycan, or presence or absence of the novel Tra-target glycan in the sample and the novel Tra-target glycan is preferably as defined in Formula I,
d) optionally modifying the cells with chemically or by altering cell culture condition
e) optionally modifying the cells under conditions specifically altering the amount of the novel Tra-antigens, preferably by β-galactosidase or sialidase or sialyltransferase treatments, preferably as described for validation methods
f) optionally repeating the assay by contacting cells with Tra antibody and measuring the complex of the antibody and the novel target glycan and optionally further reporting an amount of the novel Tra-target glycan or presence or absence of the novel Tra-target glycan in the sample.

The invention is further directed to a method or a business method for providing a service for measuring the presence or absence or the amount of the novel Tra-target glycan in a sample, preferably the sample is provided by a customer. The method preferably includes step of providing a report of the amount of the novel target glycan structure to a customer.

The invention is further preferably directed to charging or invoicing a customer for the information or the report of the amount of the novel target glycan structure in the sample. In preferred embodiment the measurement is validated by methods according to the invention. The preferred business method according to the invention includes advertising analysis of the novel target structure by a Tra-type antibody, this may be combined with other methods of the invention such as providing analysis service with regard to the novel target structure.

The report is in a preferred embodiment a written, oral or an electronic report.

### Chemical Definitions

Glycolipid and carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29).

It is assumed that Gal (galactose), Glc (glucose), GlcNAc (N-acetylglucosamine), GalNAc (N-acetylgalactosamine) and Neu5Ac are of the D-configuration, Fuc of the L-configuration, and all the monosaccharide units in the pyranose form. The amine group is as defined for natural galactos-and glucosamines on the 2-position of GalNAc or GlcNAc. Glycosidic linkages are shown partly in shorter and partly in longer nomenclature, the linkages of the sialic acid SA/Neu5X-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to type II N-acetyllactosamine, Galβ4GlcNAc, and/or type I N-acetyllactosamine, Galβ3GlcNAc and sialic acid (SA) is N-acetylneuraminic acid (Neu5Ac) or N-glycolylneuraminic acid (Neu5Gc) or any other natural sialic acid including derivatives of Neu5X. The sialic acid are referred together as NeuNX or Neu5X, wherein preferably X is Ac or Gc. Occasionally Neu5Ac/Gc/X may be referred as NeuNAc/NeuNGc/NeuNX. Term glycan means here broadly oligosaccharide or polysaccharide chains present in human or animal glycoconjugates, especially on glycolipids or glycoproteins.

Glycan epitope or epitopes means oligosaccharide sequence and elongated epitope means reducing end elongated preferred oligosaccharide sequence variants.

“Oligosaccharide sequence” means specific sequence of glycosidically linked monosaccharide residues, including terminal and “core”sequences, being oligosaccharides or glycoconjugates, In a preferred embodiment oligosaccharide sequence is “terminal oligosaccharide sequence”. The core oligosaccharide sequences can be modified by non-reducing end monosaccharide residue(s). The expression “terminal oligosaccharide sequence” indicates that the oligosaccharide is not substituted to the non-reducing end terminal residue by another monosaccharide residue or residues. Preferably the non-reducing end of the oligosaccharide sequence consist of the oligosaccharide sequence and it is only modified from the reducing end of the oligosaccharide sequence, preferably it is glycosidically conjugated from the reducing end.

It is realized that the glycan structures may be chemically conjugated to form polyvalent solid conjugates for assays or inhibition or conjugated to a solid phase for assays. Preferred conjugation types include O—, N—, C—, and S-linkages, or corresponding glycosidic linkages. Preferred conjugation type further includes oxime linkages to amino-oxy spacers or linkages to hydrazines.

The following definitions are provided for some terms used in this specification. The terms, “immunoglobulin”, “heavy chain”, “light chain” and “Fab” are used in the same way as in the European Patent Application No. 0125023.

“Antibody” in its various grammatical forms is used herein as a collective noun that refers to a population of immunoglobulin molecules and/or immunologically active portions of immunoglobulin molecules, i.e., molecules that contain an antigen binding site or a paratope. Examples of molecules which are described by the term “antibody” herein include, but are not limited to: single chain Fvs (scFvs), Fab fragments, Fab′ fragments, F(ab′) fragments, disulfide linked Fvs (sdFvs), Fvs, and fragments comprising or alternatively consisting of, either a VL or a VH domain. The immunoglobulin molecules of the invention can be of any type (e.g., IgG, IgE, IgM, IgD, IgA and IgY), or subclass of immunoglobulin molecule. Preferably, an antibody of the invention comprises, or alternatively consists of, a VH domain, VH CDR, VL domain, or VL CDR. In broadest sense the term antibody includes any polypeptide with glycan antigen binding structure, paratope, conformation binding specifically or exclusively the preferred glycan epitopes of the invention. It is realized that these can be engineered using antibody variable domain conformations and/or known linear polylactosamine binding protein structures.

An “antigen-binding site”, a “paratope”, is the structural portion of an antibody molecule that specifically binds an antigen.

Antibodies. Known methods are used for the production of antibodies, e.g any suitable host animal is immunized, antibody is expressed from cloned immunoglobulin cDNAs and/or an antibody library such as phage display library is screened, preferably against the preferred target and control saccharides of the invention e.g. as defined in WO2009060129. Monoclonal antibody preparation include hybridoma techniques (Köhler et al., Nature, 256: 495-497, 1975; Kosbor et al., Immunology Today, 4: 72, 1983; Cole et al., Monoclonal Antibodies and Cancer Therapy, Alan R Liss, Inc., pp. 77-96, 1985), all incorporated herein by reference.

Information on useful binder specifities including lectin and elongated antibody epitopes is available from reviews and monographs such as (Debray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.jp/epitope/, which list glycan specificities of monoclonal antibodies.

The three dimensional structures of the antibodies are in general known, the exact structure of the preferred antibodies of the invention comprises antigen binding variable domain sequences of heavy chain CDR1-3 and light chain CDR1-3 complementary to the three dimensional structures of the binding saccharide epitopes according to the invention. The data of inventors indicates the antibody binding is dependent on the CDR regions. The structure is defined by the glycan structures which conformations are in general well known.

The conformations of the Type I-II tetraose epitope of the invention, Galβ3GlcNAcβ3Galβ4GlcNAc-structures and control are available by standard glycan modelling e.g. as described in WO/2005/037187, WO/2001/043751 or based on similar structure e.g. from Sweetdb database, at German Cancer Research Center Heidelberg, Central Spectroscopic Division, Im Neuenheimer Feld 280, 69120 Heidelberg, Germanyand Justus-Liebig University Gielβen, Institute of Biochemistry and Endocrinology, Frankfurter Str. 100, 35392 Gielβen, Germany, (web access http://www.glycosciences.de/sweetdb/structure/) preferred analogous structures with Galβ3GlcNAcβ3Galβ4GlcNAc-epitopes with published 3D coordinates include e.g.: linucsid=12027 (http://www.glycosciences.de/sweetdb/start.php?action=explore_linucsid&linucsid=1202 7 all structures accessed Apr. 26, 2010), the tetraose, linucsid=16980 http://www.glycosciences.de/sweetdb/start.php?action=explore_linucsid&linucsid=16980, the tetraose on Core II O-glycan structure as revealed from ESC cells by the inventors; and linucsid=4453 (the tetraose linked (33 to Lac oligosaccharide) (http://www.glycosciences.de/sweetdb/start.php?action=explore_linucsid&linucsid=4453, accessed Apr. 26, 2010). Corresponding Galβ3GlcNAcβ3Galβ4Glc-epitope: linucsid=8306 (http://www.glycosciences.de/sweetdb/start.php?action=explore_linucsid&linucsid=8306, accessed Apr. 26, 2010) and PDB-database entry 2z8f, X-ray diffraction structure (http://www.pdb.ora/pdb/explore/explore.do?structureId=2z80, PDB database is managed by two members of the RCSB, the Universities Rutgers (The State University of New Jersey) and UCSD (University of California, San Diego), USA),

The complementary antibody conformation binding Galβ3GlcNAcβ3Galβ4GlcNAc- includes regions recognizing lactosamine residues including preferably i) polar, which form hydrogen bond(s) with non-reducing end type I lactosamine Galβ3GlcNAc, ii) polar amino acid residue(s) having hydrogen bonding to at least one hydroxyl group of the reducing end type II lactosamine epitope Galβ4GlcNAc iii) or/an aliphatic or aromatic amino acid residue in van der Waals contact with NAc-group on position 2 of the reducing end position GlcNAc, preferably with its methyl proton, iv) large linear binding site conformation capable of affinity increasing interaction with both lactosamine residues in linear tetrasaccharide chain. The interactions can be further defined by crystallography or molecular modelling based on antibody and glycan structures or NMR such as STD NMR experiments (Maaheimo H et al. Biochemistry 2000, 12778-88). As an example STD NMR experiments are performed with soluble oligosaccharides or monovalent conjugates as described in the publications. In a preferred embodiment the invention is directed to complex having essentially 3D structures of TRA-antibody clones of the invention, preferably Tra-1-60, or in other embodiment other engineered or natural proteins binding the epitope, preferably Galβ3GlcNAc binding antibodies, such as K21 antibody or polylactosamine binding antibody engineered to specifically to bind the epitope according to invention and less effectively Lacto-N-tetraose epitope with reducing end glucose. In preferred embodiment the invention is directed to antibody complexes giving NMR signals corresponding to interaction defined above and/or giving essentially similar NMR signals in STD NMR or the preferred antibody complexes.

Preferred protein structures for engineering optimized binding specificities for the Type I-II tetraose epitope recognition include: antibody selected from the group TRA-antibodies, engineered Galβ3GlcNAc binding antibodies such as K21-antibodies, linear β3-linked polylactosamine binding antibodies, engineered enzymes such as engineered Lacto-N-biosidase (3D, structure with LNT oligosaccharide is known PDB-database entry 2z8f) or polysaccharide cleaving enzymes: β-glucanases or glycosaminoglycan cleaving enzymes or chitinases or lysozymes or engineered forms of other polylactosamine recognizing molecules/protein such lectin as potato (STA) or tomato (LTA) lectins or wheat germ agglutinin WGA and its non-sialic acid binding succinylated form.

### EXAMPLES

### Material and Methods

Tra-1-81 and Tra-1-60 antibodies were from Chemicon (MAB4381 and MAB4361, respectively).

Glycan microarray analysis was carried out by the Consortium for Functional Glycomics. Glycan microarrays were printed as described (Blixt 2004). Version 3.2 of the human printed glycan array was used for analysis. Binding analysis was performed at 50 mg/ml of antibody. Data are reported as average RFU of 6 replicates after removal of highest and lowest values.

### Tra-1-81

The glycan microarray data reveales that Tra-1-81 binding to type 1 N-acetyllactosamine β1-3 linked to N-acetyllactos amine Galβ1-3GlcNAcβ1-3Galβ1-4GlcNAc. Tra-1-81 also weakly bound to Lewis a and or lactose derivative Galβ1-3GlcNAcβ1-3Galβ1-4Glc and blood group A1 antigen.

### Highly Specific and Unusual Tra-1-81 Epitopes

The present invention revealed especially preferred epitope structure Galβ1-3GlcNAcβ1-3Galβ1-4GlcNAc and the same epitope on branched glycan Galβ1-3GlcNAcβ1-3Galβ1-4GlcNAcβ6(Galβ4GlcNAcβ3)Galβ4Glc which were specifically bound by the Tra-1-81 antibody. The signals shown in the Table 1 were 721 and 147, respectively. The binding is very specific. The related structures including tetrasaccharides with reducing end Glc Galβ1-3GlcNAcβ1-3Galβ1-4Glc (no 131, consortium for glycobiology glycan array glycan numbering), with two type 1 N-acetyllactosamines Galβ1-3GlcNAcβ1-3Galβ1-3GlcNAc (no 280), isomer with β6-linkage with lactosamines Galβ1-3GlcNAcβ1-6Galβ1-4GlcNAc (no 403), or two type II LacNAcs Galβ1-4GlcNAcβ1-3Galβ1-4GlcNAc (no 146), variant with reducing end Glc and sialic acid on position 6 of subterminal GlcNAc Galβ1-3(Neu5Acα6)GlcNAcβ1-3Galβ1-4Glc (no 126), α3-sialylated variant Neu5Acα3Galβ1-3GlcNAcβ1-3Galβ1-4GlcNAc (no 223), sialylated and difucosylated derivative Neu5Acα3Galβ1-3(Fucα4)GlcNAcβ1-3Galβ1-4(Fucα3)GlcNAc (no 217), trisaccharide lacking non-reducing end Gal, GlcNAcβ1-3Galβ1-4GlcNAc (no 163 and 164), or non-reducing end disaccharide Galβ3GlcNAc (nos 132 and 133) had much weaker binding or practically no binding.

General methods for selecting new antibodies: antibody selection by phage display screening has been published in Jylhä S et al., WO/2008/092992, methods to select anti-glycan antibodies by phage display methods in Wang L et al., Mol Immunol. 1997 June; 34(8-9):609-18, and methods to obtain anti-glycan antibody by immunization in Galil and Repik, WO/1995/024924. The mammalian glycan array oligosaccharide codes for mammalian printed array version 3.2 are available from the Consortium for Functional Glycomics e.g. through web page http://www.functionalglycomics.org/static/consortium/resources/resourcecoreh12.shtml.

### Tra-1-60

The glycan binding specificity of Tra-1-60 is shown in Table 2. On the glycan microarray Tra-1-60 exclusively bound to type 1 N-acetyllactosamine β1-3 linked to N-acetyllactosamine (Galβ1-3GlcNAcβ1-3Galβ1-4GlcNAc) with some very weak or non-essential binding to lactose derivative Galβ1-3GlcNAcβ1-3Galβ1-4Glc.

### Highly Specific and Unusual Tra-1-60 Epitope

The present invention revealed especially preferred epitope structure Galβ1-3GlcNAcβ1-3Galβ1-4GlcNAc (number 130) and the same epitope on branched glycan Galβ1-3GlcNAcβ1-3Galβ1-4GlcNAcβ6(Galβ4GlcNAcβ3)Galβ4Glc (number 379) as antibody target structures, which were specifically bound by the Tra-1-60 antibody. The signals shown in the Table 2. The binding to these was very effective and this much stronger than binding to the similar control glycans as described for Tra-1-81, these antibodies and the corresponding antibodies are referred here as Tra-antibodies and Tra-specificity.

TABLE 1
Glycan binding specificity of Tra-1-81. 50 glycan structures giving the highest
signals out of the 406 glycan structures on the microarray.

| Chart # | Masterlist Name | RFU | STDEV |
|---|---|---|---|
| 130 | Galb1-3GlcNAcb1-3Galb1-4GlcNAcb-Sp0 | 721 | 597 |
| 379 | Galb1-3GlcNacb1-3(Galb1-3GlcNacb1-3Galb1-4GlcNacb1-6)Galb1-4Glcb-Sp0 | 147 | 64 |
| 390 | GalNAca1-3(Fuca1-2)Galb1-3GalNAca1-3(Fuca1-2)Galb1-4GlcNAcb-Sp0 | 72 | 26 |
| 118 | Galb1-3(Fuca1-4)GlcNAcb-Sp8 | 53 | 51 |
| 131 | Galb1-3GlcNAcb1-3Galb1-4Glcb-Sp10 | 34 | 24 |
| 30 | [3OSO3]Galb1-3GalNAca-Sp8 | 30 | 15 |
| 329 | GalNAca1-3(Fuca1-2)Galb1-4GlcNAcb1-3Galb1-4GlcNAcb-Sp0 | 26 | 19 |
| 365 | GalNAca1-3(Fuca1-2)Galb1-4GlcNAcb1-2Mana1-3(GalNAca1-3(Fuca1-2)Galb1-4GlcNAcb1-2Mana1-6 [symbol] | 25 | 13 |
| 315 | Neu5Aca2-3Galb1-4GlcNAcb1-2Mana1-3(Neu5Aca2-6Galb1-4GlcNAcb1-2Mana1-6)Manb1-4GlcNAcb [symbol] | 25 | 25 |
| 398 | Galb1-4(Fuca1-3)GlcNacb1-3GalNaca-Sp14 | 24 | 33 |
| 289 | Galb1-3GalNAca-Sp16 | 24 | 10 |
| 70 | Fuca1-2Galb1-4GlcNAcb1-3Galb1-4GlcNAcb1-3Galb1-4GlcNAcb-Sp0 | 23 | 5 |
| 270 | Fuca1-2Galb1-4[6OSO3]Glc-Sp0 | 23 | 13 |
| 366 | Gala1-3(Fuca1-2)Galb1-4GlcNAcb1-2Mana1-3(Gala1-3(Fuca1-2)Galb1-4GlcNAcb1-2Mana1-6)Manb1 [symbol] | 22 | 13 |
| 88 | GalNAcb1-3(Fuca1-2)Galb-Sp8 | 21 | 16 |
| 173 | GlcNAcb1-6(Galb1-3)GalNAca-Sp8 | 21 | 25 |
| 349 | Galb1-3(Fuca1-4)GlcNAcb1-2Mana1-3(Galb1-3(Fuca1-4)GlcNAcb1-2Mana1-6)Manb1-4GlcNAcb1-4Gl [symbol] | 21 | 4 |
| 370 | Fuca1-2Galb1-3(Fuca1-4)GlcNAcb1-2Mana1-3(Fuca1-2Galb1-3(Fuca1-4)GlcNAcb1-2Mana1-6)Manb1 [symbol] | 20 | 8 |
| 80 | GalNAca1-3(Fuca1-2)Galb1-4GlcNAcb-Sp0 | 20 | 11 |
| 67 | Fuca1-2Galb1-4(Fuca1-3)GlcNAcb-Sp0 | 20 | 24 |
| 384 | Galb1-4GlcNacb1-2(Galb1-4GlcNacb1-4)Mana1-3(Galb1-4GlcNacb1-2(Galb1-4GlcNacb1-6)Mana1-6) [symbol] | 20 | 27 |
| 215 | Neu5Aca2-3Galb1-3[6OSO3]GlcNAc-Sp8 | 20 | 3 |
| 45 | [6OSO3]GlcNAcb-Sp8 | 20 | 18 |
| 4 | Galb1-3GlcNAcb1-2Mana1-3(Galb1-3GlcNAcb1-2Mana1-6)Manb1-4GlcNAcb1-4GlcNAcb-Sp19 | 19 | 16 |
| 111 | Gala1-4GlcNAcb-Sp8 | 19 | 16 |
| 290 | Galb1-3(Neu5Aca2-3Galb1-4(Fuca1-3)GlcNAcb1-6)GalNAca-Sp14 | 19 | 11 |
| 225 | Neu5Aca2-3Galb1-3GlcNAcb-Sp8 | 19 | 2 |
| 284 | [3OSO3][4OSO3]Galb1-4GlcNAcb-Sp0 | 19 | 9 |
| 107 | Gala1-4(Fuca1-2)Galb1-4GlcNAcb-Sp8 | 18 | 21 |
| 126 | Galb1-3GalNAcb1-3Gala1-4Galb1-4Glcb-Sp0 | 18 | 12 |
| 202 | Neu5Aca2-8Neu5Aca2-8Neu5Aca2-8Neu5Aca2-3(GalNAcb1-4)Galb1-4Glcb-Sp0 | 18 | 11 |
| 400 | Gala1-4Galb1-3GlcNacb1-2Mana1-3(Gala1-4Galb1-3GlcNacb1-2Mana1-6)Manb1-4GlcNacb1-4GlcNa [symbol] | 18 | 27 |
| 42 | [6OSO3]Galb1-4GlcNAcb-Sp8 | 18 | 7 |
| 168 | GlcNAcb1-4(GlcNAcb1-6)GalNAca-Sp8 | 18 | 8 |
| 2 | Neu5Aca2-8Neu5Aca2-8Neu5Acb-Sp8 | 18 | 2 |
| 285 | [6OSO3]Galb1-4[6OSO3]GlcNAcb-Sp0 | 18 | 11 |
| 115 | Galb1-3(Fuca1-4)GlcNAcb1-3Galb1-4GlcNAcb-Sp0 | 17 | 8 |
| 314 | Neu5Aca2-3Galb1-3GalNAca-Sp14 | 17 | 9 |
| 194 | Mana1-3(Mana1-6)Mana-Sp9 | 17 | 11 |
| 56 | Fuca1-2Galb1-3GalNAcb1-3Gala1-4Galb1-4Glcb-Sp9 | 17 | 8 |
| 352 | KDNa2-6Galb1-4GlcNAc-Sp0 | 17 | 10 |
| 84 | GalNAca1-3GalNAcb-Sp8 | 17 | 13 |
| 244 | Neu5Aca2-6Galb1-4GlcNAcb-Sp8 | 17 | 10 |
| 324 | Neu5Aca2-6Galb1-4GlcNAcb1-3Galb1-3GlcNAcb-Sp0 | 17 | 4 |
| 167 | GlcNAcb1-4-MDPLys | 16 | 15 |
| 228 | Neu5Aca2-3Galb1-4(Fuca1-3)GlcNAcb1-3Galb1-4(Fuca1-3)GlcNAcb1-3Galb1-4(Fuca1-3)GlcNAcb-Sp [symbol] | 16 | 7 |
| 151 | Galb1-4GlcNAcb-Sp0 | 16 | 12 |
| 32 | [3OSO3]Galb1-4(Fuca1-3)GlcNAcb-Sp8 | 16 | 17 |
| 372 | NeuAca2-6Galb1-4GlcNAcb1-3GalNAc-Sp14 | 16 | 27 |
| 198 | Neu5Aca2-6Galb1-4GlcNAcb1-2Mana1-3(Neu5Aca2-3Galb1-4GlcNAcb1-2Mana1-6)Manb1-4GlcNAcb [symbol] | 16 | 6 |

[symbol]

indicates data missing or illegible when filed

TABLE 2
Glycan binding specificity of Tra-1-60. 50 glycan structures giving the highest
signals out of the 406 glycan structures on the microarray are shown.

| Chart # | Masterlist Name | RFU | STDEV |
|---|---|---|---|
| 130 | Galb1-3GlcNAcb1-3Galb1-4GlcNAcb-Sp0 | 5435 | 3990 |
| 379 | Galb1-3GlcNacb1-3(Galb1-3GlcNacb1-3Galb1-4GlcNacb1-6)Galb1-4Glcb-Sp0 | 3910 | 1754 |
| 118 | Galb1-3(Fuca1-4)GlcNAcb-Sp8 | 57 | 95 |
| 228 | Neu5Aca2-3Galb1-4(Fuca1-3)GlcNAcb1-3Galb1-4(Fuca1-3)GlcNAcb1-3Galb1-4(Fuca1-3)GlcNAcb-Sp [symbol] | 38 | 12 |
| 349 | Galb1-3(Fuca1-4)GlcNAcb1-2Mana1-3(Galb1-3(Fuca1-4)GlcNAcb1-2Mana1-6)Manb1-4GlcNAcb1-4Gl [symbol] | 33 | 15 |
| 30 | [3OSO3]Galb1-3GalNAca-Sp8 | 32 | 20 |
| 366 | Gala1-3(Fuca1-2)Galb1-4GlcNAcb1-2Mana1-3(Gala1-3(Fuca1-2)Galb1-4GlcNAcb1-2Mana1-6)Manb1- [symbol] | 29 | 21 |
| 365 | GalNAca1-3(Fuca1-2)Galb1-4GlcNAcb1-2Mana1-3(GalNAca1-3(Fuca1-2)Galb1-4GlcNAcb1-2Mana1-6 [symbol] | 27 | 11 |
| 18 | GalNAcb-Sp8 | 27 | 10 |
| 374 | GalNAcb1-4GlcNAcb1-2Mana1-6(GalNAcb1-4GlcNAcb1-2Mana1-6)Manb1-4GlcNAcb1-4GlcNAc-Sp12 [symbol] | 24 | 5 |
| 363 | Fuca1-2Galb1-3GlcNAcb1-3(Galb1-4(Fuca1-3)GlcNAcb1-6)Galb1-4Glc-Sp21 | 22 | 6 |
| 336 | GlcNAca1-4Galb1-4GlcNAcb1-3Galb1-4(Fuca1-3)GlcNAcb1-3Galb1-4(Fuca1-3)GlcNAcb-Sp0 | 22 | 16 |
| 381 | Fuca1-2Galb1-3(Fuca1-4)GlcNAcb1-3(Galb1-4GlcNAcb1-6)Galb1-4Glc-Sp21 | 21 | 9 |
| 214 | Neu5Aca2-3GalNAcb1-4GlcNAcb-Sp0 | 21 | 18 |
| 392 | Gala1-3Galb1-3(Fuca1-4)GlcNAcb1-2Mana1-3(Gala1-3Galb1-3(Fuca1-4)GlcNAcb1-2Mana1-6)Manb1- [symbol] | 20 | 11 |
| 317 | Neu5Aca2-6Galb1-4GlcNAcb1-2Mana1-3(GlcNAcb1-2Mana1-6)Manb1-4GlcNAcb1-4GlcNAcb-Sp12 | 20 | 5 |
| 43 | [6OSO3]Galb1-4[6OSO3]Glcb-Sp8 | 20 | 17 |
| 358 | Gala1-3Galb1-4GlcNAcb1-2Mana1-3(Gala1-3Galb1-4GlcNAcb1-2Mana1-6)Manb1-4GlcNAcb1-4GlcN [symbol] | 20 | 21 |
| 233 | Neu5Aca2-3Galb1-4GlcNAcb1-3Galb1-4(Fuca1-3)GlcNAc-Sp0 | 20 | 24 |
| 226 | Neu5Aca2-3Galb1-4[6OSO3]GlcNAcb-Sp8 | 20 | 6 |
| 373 | Neu5Aca2-3Galb1-4(Fuca1-3)GlcNAcb1-3GalNAca-Sp14 | 20 | 7 |
| 274 | Galb1-3(Neu5Aca2-3Galb1-4GlcNacb1-6)GalNAca-Sp14 | 20 | 12 |
| 203 | Neu5Aca2-8Neu5Aca2-8Neu5Aca2-3(GalNAcb1-4)Galb1-4Glcb-Sp0 | 19 | 16 |
| 104 | Gala1-3Galb1-4GlcNAcb-Sp8 | 18 | 12 |
| 337 | GlcNAca1-4Galb1-4GlcNAcb1-3Galb1-4GlcNAcb-Sp0 | 18 | 10 |
| 330 | GalNAca1-3(Fuca1-2)Galb1-4GlcNAcb1-3Galb1-4GlcNAcb1-3Galb1-4GlcNAcb-Sp0 | 17 | 26 |
| 215 | Neu5Aca2-3Galb1-3[6OSO3]GlcNAc-Sp8 | 17 | 12 |
| 131 | Galb1-3GlcNAcb1-3Galb1-4Glcb-Sp10 | 17 | 11 |
| 352 | KDNa2-6Galb1-4GlcNAc-Sp0 | 17 | 4 |
| 342 | Neu5Aca2-6Galb1-4GlcNAcb1-2Mana1-3Manb1-4GlcNAcb1-4GlcNAc-Sp12 | 17 | 15 |
| 290 | Galb1-3(Neu5Aca2-3Galb1-4(Fuca1-3)GlcNAcb1-6)GalNAca-Sp14 | 17 | 10 |
| 249 | Neu5Aca2-6Galb-Sp8 | 16 | 15 |
| 209 | Neu5Aca2-3(GalNAcb1-4)Galb1-4GlcNAcb-Sp8 | 16 | 12 |
| 10 | Fuca-Sp9 | 16 | 13 |
| 300 | GlcAb1-3GlcNAcb-Sp8 | 15 | 14 |
| 278 | Galb1-4(Fuca1-3)[6OSO3]Glc-Sp0 | 15 | 13 |
| 353 | KDNa2-3Galb1-4Glc-Sp0 | 15 | 9 |
| 64 | Fuca1-2Galb1-3GlcNAcb-Sp8 | 15 | 8 |
| 302 | GlcNAcb1-2Mana1-3(GlcNAcb1-2Mana1-6)Manb1-4GlcNAcb1-4GlcNAcb-Sp12 | 15 | 11 |
| 1 | Neu5Aca2-8Neu5Acb-Sp17 | 15 | 18 |
| 401 | Gala1-4Galb1-4GlcNacb1-2Mana1-3(Gala1-4Galb1-4GlcNacb1-2Mana1-6)Manb1-4GlcNacb1-4GlcNa [symbol] | 15 | 10 |
| 264 | [3OSO3]Galb1-4(Fuca1-3)[6OSO3]GlcNAc-Sp8 | 14 | 8 |
| 362 | Neu5Aca2-6GlcNAcb1-4GlcNAcb1-4GlcNAc-Sp21 | 14 | 14 |
| 384 | Galb1-4GlcNacb1-2(Galb1-4GlcNacb1-4)Mana1-3(Galb1-4GlcNacb1-2(Galb1-4GlcNacb1-6)Mana1-6) [symbol] | 14 | 3 |
| 173 | GlcNAcb1-6(Galb1-3)GalNAca-Sp8 | 14 | 4 |
| 162 | GlcNAcb1-3Galb1-3GalNAca-Sp8 | 14 | 5 |
| 96 | Gala1-3(Fuca1-2)Galb1-4GlcNAc-Sp0 | 14 | 21 |
| 152 | Galb1-4GlcNAcb-Sp8 | 14 | 8 |
| 48 | Mana1-3(Mana1-6)Manb1-4GlcNAcb1-4GlcNAcb-Sp13 | 14 | 8 |
| 268 | Fuca1-2[6OSO3]Galb1-4[6OSO3]Glc-Sp0 | 14 | 10 |

[symbol]

indicates data missing or illegible when filed

1.-20. (canceled)

21. A complex of an antibody and an isolated glycan comprising a target structure epitope according to Formula 1
Galβ1-3GlcNAcβ1-3Galβ1-GlcNAc. (1)

22. The complex according to claim 21, wherein said antibody is not essentially capable of binding to structure Galβ1-3GlcNAcβ1-3Galβ1-Glc.

23. The complex according to claim 21, wherein said antibody is not essentially capable of binding to the structure according to Formula 2
(T)ₚGalβ1-3(Fucα4)ₙGlcNAcβ1-3Galβ1-Glc(NAc)ₘR, (2)
wherein p, n and m are integers 0 or 1 independently and/or the larger reducing end elongated or conjugated target oligosaccharide sequences thereof, and wherein the fucose residue in (2) indicated by n is a branch in the structure, and T is terminal monosaccharide residue including a sialic acid, and R is reducing end derivative or conjugate as defined above with the proviso that when m is 1 either p or n is also 1 and the structure is not the binding epitope Galβ1-3GlcNAcβ1-3Galβ1-4GlcNAc.

24. The complex according to claim 21, wherein said antibody is not essentially capable of binding to the structure according to Formula 3
(T)ₚGalβ1-z(Fucα4)ₙGlcNAcβ1-yGalβ1-qGlc(NAc)ₘR, (3)
wherein p, n and m are integers 0 or 1 independently and/or the larger reducing end elongated or conjugated target oligosaccharide sequences thereof, and wherein the fucose residue in (3) indicated by n is a branch in the structure, and T is terminal monosaccharide residue including a sialic acid; and z, y and q are linkage positions selected from the group 3, 4, or 6, independently, and y is 3 or 6, with the proviso that when m is 1, and z and y are 3, and q is 4, either p or n is also 1.

25. The complex according to claim 21, wherein said complex is in an array of glycan structures, and optionally the array comprises said saccharides the antibody is capable of binding and optionally further said saccharides the antibody is not capable of binding.

26. The complex in a form of array of claim 25, wherein the array comprises Galβ1-3GlcNAcβ1-3Galβ1-GlcNAc and saccharide Galβ1-3GlcNAcβ1-3Galβ1-Glc or reducing end conjugates or derivatives thereof.

27. The complex in a form of array of claim 25, wherein said antibody is not essentially capable of binding to the structure according to Formula 2
(T)ₚGalβ1-3(Fucα4)ₙGlcNAcβ1-3Galβ1-Glc(NAc)ₘR, (2)
wherein p, n and m are integers 0 or 1 independently and/or the larger reducing end elongated or conjugated target oligosaccharide sequences thereof, and wherein the fucose residue in (2) indicated by n is a branch in the structure, and T is terminal monosaccharide residue including a sialic acid, and R is reducing end derivative or conjugate as defined above with the proviso that when m is 1 either p or n is also 1 and the structure is not the binding epitope Galβ1-3GlcNAcβ1-3Galβ1-4GlcNAc.

28. The complex according to claim 27, wherein said glycan array is a solid phase conjugated saccharide array.

29. A method of using of the complex and/or target saccharides according to claim 21 in an assay including a solid phase assay or liquid phase assay involving binding of the antibody to the oligosaccharide glycan or chemical synthetic conjugate of the glycan.

30. The method according to claim 29 for the screening a new antibody with Tra-specificity, said method comprising a step of contacting a sample containing antibodies with said target saccharide.

31. The method according to claim 30, wherein the method includes steps of
a. providing a sample comprising at least one antibody or functional antibody fragment binding to an antigen;
b. contacting the sample with a glycan structure comprising terminal non-reducing end terminal oligosaccharide sequence according to the Formula 1
Galβ1-3(Fucα4)_{d}GlcNAcβ1-3Galβ1-Glc(NAc)ₘ, (1)
wherein n and m are integers 0 or 1 respectively;
c. measuring the binding of the antibody to the oligosaccharide sequence;
d. optionally contacting the antibody sample with at least one control glycan structure;
e. optionally selecting antibody with specific binding to the target structures but low or non-existent binding to specificity control saccharides, or in a specific embodiment selecting antibodies with additionally or specifically corresponding Lewis a specificity;
f. optionally using an oligosaccharide sequence comprising the terminal non-reducing end saccharide sequence of Formula 1 or being the saccharide for the inhibition of the binding of the antibody to the oligosaccharide sequence; and
g. optionally using β3-galactosidase of α3- and or α6-sialidase enzymes to optimize or reduce the amount of the antibody target structures on cells.

32. The method according to claim 30, for production of Lewis a variants of Tra-antibodies wherein m is 1 and n is 0 or 1; or for production of LNT-type variants wherein m is 0 or 1, and n is 1.

33. The method according to claim 30 for optimization of the binding activity of a Tra-type antibody using the saccharide sequence of Formula 1b.

34. The method according to claim 29, wherein the tetrasaccharide comprising control material or said complex is used for validation of the analysis of the antibody binding to cells or other biological materials.

35. The method according to claim 30, wherein the method further involves a control material, which comprises a purified oligosaccharide according to Formula 1 or its chemical conjugate or natural or biosynthetic material enriched with regard to the oligosaccharide sequence.

36. The method according to claim 30, wherein the saccharide epitope is conjugated to a solid surface or to control cells in a solid phase assay or used as a soluble inhibitor or soluble analyst (e.g. labelled conjugate for a fluorescence polarization assay) to validate the binding specificity of the antibody.

37. A Tra-antibody analysis kit comprising the saccharide sequence of Formula 1b comprising glycan or glycoconjugate or a cell sample optimized with the glycan structure expression and/or instructions for the analysis of, and optionally instructions for reporting the amount of the novel target structure.

38. The method according to claim 29, involving use of a specific α3- (or α6-)sialidase enzyme to optimize the presence of the Tra antigens on cell surface and/and a specific β3-galactosidase is used to reduce the amount of the structure on cells.

39. The method according to claim 29 involving further use of the produced antibodies for the analysis of stem cells or cancer cells or other cells or tissues known to bind to Tra-antibodies including human embryonic type stem cells and pluripotent equivalents thereof such as IPS cells, induced pluripotent cells or mesenchymal stem cells or osteogenically or adipocyte differentiated mesenchymal stem cells.

40. The method according to claim 29 for assay of biological materials including the step of reporting of the amount of novel target glycan according to Formula 1, including the steps of:
a) providing a biological material;
b) contacting the material with a Tra-antibody;
c) providing a report indicating an amount of the novel Tra-target glycan, or presence or absence of the novel Tra-target glycan in the sample and the novel Tra-target glycan is as defined in Formula 1;
d) optionally modifying the cells with chemically or by altering cell culture condition;
e) optionally modifying the cells under conditions specifically altering the amount of the novel Tra-antigens by β-galactosidase or sialidase or sialyltransferase treatments;
f) optionally repeating the assay by contacting cells with Tra antibody and measuring the complex of the antibody and the novel target glycan and optionally further reporting an amount of the novel Tra-target glycan or presence or absence of the novel Tra-target glycan in the sample.
